(19) United States
(12) Patent Application Publication
(10) Pub. No.: US 2009/0253129 A1
(43) Pub. Date: Oct. 8, 2009

(54) IDENTIFICATION OF USA300 COMMUNITY-ASSOCIATED METHICILLIN-RESISTANT STAPHYLOCOCCUS AUREUS

(75) Inventor: Richard V. Goering, Omaha, NE (US)

Correspondence Address: MUETING, RAASCH & GEBHARDT, P.A., P.O. BOX 581336, MINNEAPOLIS, MN, 55458-1336, US

(73) Assignee: CREIGHTON UNIVERSITY, Omaha, NE (US)

(21) Appl. No.: 12/083,917

(22) PCT Filed: Nov. 9, 2006
(86) PCT No.: PCT/US2006/043780

§ 371 Date: Nov. 24, 2008

Related U.S. Application Data
(60) Provisional application No. 60/736,147, filed on Nov. 10, 2005.
(60) Provisional application No. 60/816,803, filed on Jun. 27, 2006.

(51) Int. Cl. C12Q 1/68; C07H 21/04

(52) U.S. Cl. 435/6; 536/23.1; 536/24.33

(57) ABSTRACT

The present invention provides methods for identifying the US A300 strain of methicillin-resistant Staphylococcus aureus (MRSA).

### CONTINUING APPLICATION DATA

This application claims the benefit of U.S. Provisional Application Ser. Nos. 60/736,147, filed Nov. 10, 2005, and 60/816,803, filed Jun. 27, 2006, each of which is incorporated by reference herein.

### BACKGROUND

Community-acquired methicillin resistant Staphylococcus aureus (CA-MRSA) infections are now a cause of major clinical concern. Although first identified in the United States among intravenous drug users (Saravolatz et al., Ann Intern Med, 1982; 97:325-329), followed by other high-risk populations, such as prison inmates and athletes, hospitals nationwide have noted an increasing trend in the number of CA-MRSA infections seen in young, healthy populations without pre-disposing risk factors (Centers for Disease Control and Prevention, Morb Mortal Wkly Rep, 2003; 52:793-795; Centers for Disease Control and Prevention, Morb Mortal Wkly Rep, 2003; 52:992-996; Francis et al., Clin Infect Dis, 2005; 40:100-107; Kazakova et al., N Engl J Med, 2005; 352:468-475; Lindenmayer et al., Arch Intern Med, 1998; 158:895-899; Naimi et al., JAMA 2003; 290:2976-2984; and Saravolatz et al., Ann Intern Med 1982; 97:325-329). A prospective cohort study published by Naimi et al. found the median age of patients with CA-MRSA infections to be significantly younger than those with nocosomial-acquired MRSA, at thirty years versus seventy years., respectively (Naimi et al., JAMA 2003; 290:2976-2984). Numerous studies have also indicated that CA-MRSA infections are frequently seen among infants and children, again suggesting that the likelihood of contracting such infections is no longer limited to traditional at-risk populations (Buckingham et al., Pediatr Infect Dis J 2004; 23:619-624; Centers for Disease Control and Prevention, Morb Mortal Wkly Rep 1999; 48:707-710; Fridkin et al., N Engl J Med 2005; 352:1436-1444; Herold et al., JAMA 1998; 279:593-598; Mishaan et al., Pediatr Infect Dis J 2005; 24:201-206).

CA-MRSA strains commonly harbor the SCCmec type IV element and are susceptible to multiple non-β-lactam antibiotics. This is in contrast to healthcare-associated strains such as USA100 isolates, which carry the SCCmec type II element and are resistant to a wide range of antibiotics due to the presence of multiple mobile and non-mobile genetic elements (McDougal et al., J Clin Microbiol, 2003; 41:5113-5120). However, community-acquired strains typically carry the Panton-Valentine Leukociden (PVL) genes, lukS and lukF, which produce cytotoxins that cause leukocyte destruction and tissue necrosis (Genestier et al., J Clin Invest, 2005; 115:3117-3127). Strains producing PVL have been associated with skin abscess formation, furunculosis, and severe cases of necrotizing pneumonia (Lina et., Clin Infect Dis, 1999; 29:1128-1132). The presence of PVL genes may also be associated with increased disease severity (Chambers, N Engl J Med, 2005; 352:1485-1487; and Etienne, Clin Infect Dis, 2005; 41:591-593). Despite their community-acquired designation, CA-MRSA strains are frequently isolated from and transmitted among patients within the hospital setting (Saiman et al., Clin Infect Dis, 2003; 37:1313-1319). CA-MRSA have also been associated with increased patient morbidity and mortality, costly treatment, and extensive eradication procedures, which underscores the value of active surveillance for the presence of these strains (Rubin et al., Emerg. Infect Dis, 1999; 5:9-17).

Molecular typing methods used to characterize MRSA strains include pulsed-field gel electrophoresis (PFGE), multi-locus sequence typing (MLST), and polymerase-chain reaction (PCR) amplification of target genes (Shopsin and Kreiswirth, Emerg. Infect Dis, 2001; 7:323-326). By PFGE, CA-MRSA isolates in the United States have, thus far, been classified as pulsed-field types (PFTs) USA300 (ST8), USA400 (ST1) (McDougal et al., J Clin Microbiol, 2003; 41:5113-5120), USA1000 (ST59), and USA1100 (ST30) by the Centers for Disease Control and Prevention (McDougal et al. 2005 Abstr. 105th Annu. Meeting Amer. Soc. Microbiol., abstr. C-043). S. aureus MW2, responsible for fatal infections in four children from North Dakota and Minnesota between 1997-1999, is considered the prototype community-acquired MRSA strain belonging to the USA400 PFT (Centers for Disease Control and Prevention, Morb Mortal Wkly Rep, 1999; 48:707-710). However, recent years have seen an alarming rise in the number of USA300 isolates identified in a variety of community populations, including children, sports participants, prisoners, military recruits, and men who have sex with men (Begier et al., Clin Infect Dis 2004; 39:1446-1453; Buckingham et al., Pediatr Infect Dis J, 2004; 23:619-6243; Gonzalez et al., Pediatrics 2005; 115:642-648; Hidron et al., Clin Infect Dis 2005; 41:159-166; Miller et al., N Engl J Med, 2005; 352:1445-1453; Nguyen et al., Emerg Infect Dis, 2005; 11:526-532; and Pan et al., J Infect Dis 2005; 192:811-818). Detection of USA300 CA-MRSA strains has traditionally required the use of PFGE, MLST, and PCR (i.e, PVL) which, taken together, are time consuming and require equipment that may not be readily available to the routine clinical laboratory. In addition, the newly described arginine catabolic mobile element (ACME) (Diep et al., Lancet 2006; 367:731-739) appears to only be present in USA300 strains carrying SCCmec type IVa (McDougal et al., Abstr. 46th 2 Intersci. Conf. Antimicrob. Agents Chemother., abstr. C2-603, 2006). Thus, there is a need for a more unified molecular approach to the rapid identification of USA300 CA-MRSA isolates.

### SUMMARY OF THE INVENTION

The present invention includes a method for identifying the USA300 strain of methicillin-resistant S. aureus, the method including analyzing a methicillin-resistant S. aureus bacterium for the presence or absence of a gene for the Panton-Valentine Leukocidin (PVL) toxin; and analyzing methicillin-resistant S. aureus DNA for the presence or absence of an AT repeat region in the conserved hypothetical gene SACOL0058; wherein the presence of a gene for the PVL toxin and the presence of an AT repeat region having at least 6 AT repeats in the conserved hypothetical gene SACOL0058 indicates that the methicillin-resistant S. aureus bacterium is the USA 300 strain of methicillin-resistant S. aureus.

In some embodiments of the method, analyzing methicillin-resistant S. aureus DNA for the presence or absence of an AT repeat region in the conserved hypothetical gene SACOL0058 includes performing a polymerase chain reaction with oligonucleotide primers capable of amplifying an AT repeat region in the conserved hypothetical gene SACOL0058 if present; and analyzing for the presence or absence of amplified DNA fragments containing an AT repeat region. In some embodiments, the oligonucleotide primers will only amplify the AT repeat region when 6 or more AT repeats are present. In some embodiments, the oligonucleotide primers is a forward primer having one or more locked nucleic acid bases incorporated therein. In some embodiments, the forward oligonucleotide primer has the sequence TGCTCGACGTCAATATATATATAT (SEQ ID NO:7), wherein one or more of the nucleic acids is a locked nucleic acid base. In some embodiments, the forward oligonucleotide primer has the sequence G^{L}CT^{L}CGA^{L}CGTCAA^{L}TA^{L}T ATATATAT (SEQ ID NO:5) wherein N^{L} represents a locked nucleic acid base. In some embodiments, the oligonucleotide primers include a reverse primer having the sequence 5′-ACGATGATATTCCCGATAG-3′ (SEQ ID NO:8) or 5′-CAATTAACGATGATATTCCCGATAG-3′ (SEQ ID NO:4). In some embodiments, the method further includes sequencing the amplified DNA fragments.

In some embodiments of the method, analyzing a methicillin-resistant S. aureus bacterium for the presence or absence of a gene for the PVL toxin includes performing a polymerase chain reaction with oligonucleotide primers capable of amplifying a gene for the PVL toxin; and analyzing for the presence or absence of amplified DNA fragments of a gene for the PVL toxin. In some embodiments, the oligonucleotide primers capable of amplifying a gene for the PVL toxin include 5′-ATCATTAGGTAAAATGTCTGGACATGATCCA-3′ (SEQ ID NO:1) and 5′-GCATCAACTGTATTGGATAGCAAAAGC-3′ (SEQ ID NO:2). In some embodiments, the method further includes sequencing the amplified DNA fragments.

In some embodiments of the method, a methicillin-resistant S. aureus bacterium other than the USA300 strain has less than 6 AT repeats and/or no gene for the PVL toxin.

In some embodiments of the method, analyzing a methicillin-resistant S. aureus bacterium for the presence or absence of a gene for the Panton-Valentine Leukocidin (PVL) toxin and analyzing methicillin-resistant S. aureus DNA for the presence or absence of an AT repeat region occurs in a single-vessel experiment.

In some embodiments of the method, analyzing methicillin-resistant S. aureus DNA for the presence or absence of an AT repeat region in the conserved hypothetical gene SACOL0058 includes performing a polymerase chain reaction with oligonucleotide primers capable of amplifying an AT repeat region in the conserved hypothetical gene SACOL0058 if present; and analyzing for the presence or absence of amplified DNA fragments containing an AT repeat region; wherein analyzing a methicillin-resistant S. aureus bacterium for the presence or absence of a gene for the PVL toxin includes performing a polymerase chain reaction with oligonucleotide primers capable of amplifying a gene for the PVL toxin; and analyzing for the presence or absence of amplified DNA fragments of a gene for the PVL toxin. In some embodiments, the method further includes analyzing methicillin-resistant S. aureus DNA for the presence or absence of the conserved hypothetical gene SACOL0058, wherein analyzing methicillin-resistant S. aureus DNA for the presence or absence of the conserved hypothetical gene SACOL0058 includes performing a polymerase chain reaction with oligonucleotide primers capable of amplifying at least a portion of the conserved hypothetical gene SACOL0058 and analyzing for the presence or absence of amplified DNA fragments containing at least a portion of the conserved hypothetical gene SACOL0058. In some embodiments, the polymerase chain reactions occur in a single-vessel experiment.

The present invention also includes an isolated DNA fragment of a methicillin-resistant S. aureus bacterium genome, wherein the fragment includes an AT repeat region that includes 6 or more AT repeats, and wherein the fragment maps to a location about 1.4 kb beyond the J1 SCCmec-chromosomal junction. In some embodiments, there are 6-8 AT repeats in the AT repeat region.

The present invention also includes an isolated DNA fragment of a methicillin-resistant S. aureus bacterium gene, wherein the fragment includes an AT repeat region having 6 or more AT repeats, and wherein the fragment includes a region corresponding to nucleotides 69954 to 70855 of S. aureus strain COL (Genebank Accession Number CP000046). In some embodiments, there are 6-8 AT repeats in the AT repeat region.

The present invention also includes an isolated oligonucleotide primer having the sequence TG^{L}CT^{L}CGA^{L}CGTCAA^{L}TA^{L}TATATATAT (SEQ ID NO:5) wherein N^{L} represent a locked nucleic acid base.

The present invention also includes an isolated oligonucleotide primer selected from the group consisting of 5′-ACGATGATATTCCCGATAG-3′ (SEQ ID NO:3) and 5′-CAATTAACGATGATATTCCCGATAG-3′ (SEQ ID NO:4).

The present invention also includes a kit with an oligonucleotide primer pair capable of amplifying an AT repeat region in the conserved hypothetical gene SACOL0058 of S. aureus DNA and an oligonucleotide primer capable of amplifying a gene for the PVL toxin. In some embodiments, the kit also includes a primer pair capable of amplifying at least a portion of the conserved hypothetical gene SACOL0058.

Unless otherwise specified, “a,” “an,” “the,” and “at least one” are used interchangeably and mean one or more than one.

### BRIEF DESCRIPTION OF THE FIGURES

FIG. 1 is a diagram showing the location of the multiplex PCR primers with reference to S. aureus strain COL (accession number CP000046). Black represents the SCCmec resistance element. “DR” represents the 3′ direct repeat region flanking the SCCmec element. Gray represents the 3.3 kb chromosomal sequence 3′ SCCmec, including SACOL0058 and the AT repeat sequence. PCR amplification using primers lnaAT (nucleotides 69490-69511) and ATreg-2 (nucleotides 70831-70855) indicates the presence of ≧6 AT repeats. SACOL0058 is detected using ATreg-1 (nucleotides 69954-69977) and Atreg-2. Amplification of the PVL genes occurs at a separate location within the chromosome. The lower black box demonstrates hybridization of the lnaAT primer (SEQ ID NO:5) with the AT repeat sequence as it would occur in a USA300 MRSA strain (SEQ ID NO:6).

FIGS. 2A-2B show PCR assays of USA300 MRSA strains. FIG. 2A shows locked nucleic acid primer identification of USA300 MRSA strains. Lane 1, 1 kb DNA ladder as a molecular size standard; lanes 2 and 3 USA300:ST8 strains CRG-1130 and CRG-1128, containing 8 and 6 AT repeats, respectively; lane 4 strain CRG-930 (USA500:ST250) containing 5 AT repeats. Lane 5, negative control. FIG. 2B shows multiplex PCR assay differentiates USA300 strains from other MRSA. Lane 1, 1 kb DNA ladder as a molecular size standard; lanes 2 and 3, USA300:ST8 strains CRG-1130 and CRG-1128, containing 8 and 6 AT repeats, respectively, as well as SACOL0058 and PVL (top band contains the LNA primer amplification product (1,365 bp), middle band contains the hypothetical protein SACOL0058 product (933 bp), and the lower band contains the PVL genes product (433 bp); lane 4, strain CRG-1112 (mecA-negative) containing 5 AT repeats; lane 6, MW2 (CC1:ST1), SACOL0058 negative, PVL; lane 7, negative control.

FIG. 3 present the nucleotide sequence (SEQ ID NO:9) and hypothetical protein sequence (SEQ ID NO: 10) of the conserved hypothetical SACOL0058 gene.

### DETAILED DESCRIPTION OF ILLUSTRATIVE EMBODIMENTS OF THE INVENTION

The present invention provides methods for identifying the USA300 strain of methicillin-resistant Staphylococcus aureus (MRSA). Staphylococcus aureus, also referred to herein as “S. aureus,” “staph,” or “staph A,” is a Gram-positive bacterium that causes a variety of infections in humans, ranging from superficial skin lesions (such as boils, styes and furunculosis), to more serious infections (such as pneumonia, mastitis, phlebitis, meningitis, and urinary tract infections), and deep-seated infections (such as osteomyelitis and endocarditis). Methicillin is a beta-lactam antibiotic. It was previously used to treat infections caused by susceptible Gram-positive bacteria, including S. aureus, that would otherwise be resistant to most penicillins, but is no longer clinically used. Its role in therapy has been largely replaced by other antibiotics, such as flucloxacillin and dicloxacillin. However, the term methicillin-resistant Staphylococcus aureus (MRSA) continues to be used to describe S. aureus strains resistant to the commonly used penicillin-related antibiotics.

The USA300 strain of methicillin-resistant S. aureus (MRSA) is a major source of community-acquired methicillin resistant Staphylococcus aureus (CA-MRSA). CA-MRSA infections have become a cause of major clinical concern. Although first identified in the United States among intravenous drug users (Saravolatz et al., Ann Intern Med, 1982; 97:325-329), followed by other high-risk populations, hospitals nationwide have noted an increasing trend in the number of CA-MRSA infections seen in young, healthy populations without pre-disposing risk factors (Centers for Disease Control and Prevention, Morb Mortal Wkly Rep, 2003; 52:793-795; Centers for Disease Control and Prevention, Morb Mortal Wkly Rep, 2003; 52:992-996; Francis et al., Clin Infect Dis, 2005; 40:100-107; Kazakova et al., N Engl J Med, 2005; 352:468-475; Lindenmayer et al., Arch Intern Med, 1998; 158:895-899; Naimi et al., JAMA, 2003; 290:2976-2984; and Saravolatz et al., Ann Intern Med, 1982; 97:325-329). Despite the community acquired designation, CA-MRSA stains are also acquired from other sources. For example, CA-MRSA strains are frequently isolated form and transmitted among patients within the hospital setting. The methods of the present invention may be used for the identification and diagnosis of the USA300 strain of MRSA, including CA-MRSA. With the present invention, S. aureus may be identified by standard microbiologic methods, such as, for example, colony and microscopic morphology, coagulase testing, or agglutination. Antimicrobial susceptibility may also be determined by standard microbiologic methods. See, for example, NCCLS, “Performance standards for antimicrobial susceptibility testing; fourteenth informational supplement,” NCCLS document M100-S14, Wayne, Pa.: NCCLS, 2004.

S. aureus strain USA300 is a methicillin-resistant strain first isolated in 2000. The complete genome sequence of USA300 CA-MRSA is known (Diep et al, Lancet 2006; 367:731-9). It harbors one circular chromosome and three plasmids. It is more virulent than S. aureus (strain COL) and highly invasive of major organs. It is also more resistant to killing by human polymorphonuclear leucocytes and causes greater host cell lysis. USA300 and COL are related by vertical descent from a common ancestor. Resistance to beta lactams and ciprofloxacin are chromosomally encoded. All unique genes in USA300 are clustered in five novel allotypes of mobile genetic elements that encode virulence or resistance determinants. The first two genetic elements are the SCCmec IV element and ACME. The third genetic element is a novel staphylococcal pathogenicity island, SaPI5, that encodes two enterotoxins closely related to SEQ and SEK in COL. The fourth genetic element is prophage phiSA2usa, which carries the genes coding for the Panton-Valentine leucocidin. The fifth genetic element is prophage phiSa3usa, which encodes staphylokinase and a chemotaxis inhibiting protein. See the worldwide web at expasy.org/sprot/hamap/STAA3.html.

The present invention includes methods for identifying the USA300 strain of MRSA by analyzing a methicillin-resistant S. aureus bacterium for the presence or absence of a gene for the Panton-Valentine Leukocidin (PVL) toxin and analyzing methicillin-resistant S. aureus DNA for the presence or absence of an AT repeat region in the conserved hypothetical gene SACOL0058, wherein the presence of a gene for the PVL toxin and the presence of an AT repeat region having at least 6 AT repeats in the conserved hypothetical gene SACOL0058 indicates that the methicillin-resistant S. aureus bacterium is the USA300 strain of methicillin-resistant S. aureus. Only USA300 isolates contain 6 or more AT repeats as well as the gene for the PVL toxin. While other MRSA isolates, such as USA500 (ST8), may exhibit ≧6 AT repeats, this is not in combination with PVL. And, while other isolates (e.g., ST80) encode PVL, this is in combination with less than 6 AT repeats. Thus, the combined detection of these elements provides for the quick and specific identification of USA300 MRSA.

Community-acquired MRSA strains typically carry the Panton-Valentine Leukociden (PVL) genes, lukS and lukF, which produce cytotoxins that cause leukocyte destruction and tissue necrosis (Genestier et al., J Clin Invest, 2005; 115:3117-3127). Strains producing PVL have been associated with skin abscess formation, furunculosis, and severe cases of necrotizing pneumonia (Lina et al., Clin Infect Dis, 1999; 29:1128-1132). The presence of PVL genes may also be associated with increased disease severity (Chambers, N Engl J Med, 2005; 352:1485-1487; and Etienne, Clin Infect Dis, 2005; 41:591-593). Despite their community-acquired designation, CA-MRSA strains are frequently isolated from and transmitted among patients within the hospital setting (Saiman et al., Clin Infect Dis, 2003; 37:1313-1319). CA-MRSA have also been associated with increased patient morbidity and mortality, costly treatment, and extensive eradication procedures, which underscores the value of active surveillance for the presence of these strains (Rubin et al., Emerg Infect Dis, 1999; 5:9-17).

The methods of the present invention include analyzing a methicillin-resistant S. aureus bacterium for the presence or absence of a gene for the Panton-Valentine Leukocidin (PVL) toxin. Any of a variety of techniques may be used to analysis a methicillin-resistant S. aureus bacterium for the presence or absence of a gene for the Panton-Valentine Leukocidin (PVL) toxin. For example, nucleic acid based hybridization assays, such as PVL EVIGENE™ (catalog No. KT104, AdvanDx, Woburn, Mass.), polymerase chain reaction (PCR) based assays (see, for example, Example 1, Jarraud et al., Infect Immun 2002: 70:631-641; and Lina et., Clin Infect Dis, 1999; 29:1128-1132), or sequencing based assays.

In a preferred embodiment, analyzing a methicillin-resistant S. aureus bacterium for the presence or absence of a gene for the PVL toxin is undertaken by performing PCR with oligonucleotide primers capable of amplifying a gene for the PVL toxin and analyzing for the presence or absence of amplified DNA fragments of a gene for the PVL toxin. In some embodiments, the resultant amplified DNA product(s) may be sequenced. In one embodiment, the oligonucleotide primers used may be luk-PV-1, 5′-ATCATTAGGTAAAATGT CTGGACATGATCCA-3′ (SEQ ID NO:1) and luk-PV-2, 5′-GCATCAACTGT ATTGGATAGCAAAAGC-3′ (SEQ ID NO:2).

Other PVL probes and primers may be used. For example, PVL probes and primers may be designed using Primer Express (ver. 2.0; Applied Biosystems, Mississauga, Ontario, Canada) and Oligo 6 (ver. 6.6.7.0; Molecular Biology Insights, Inc., Cascade, Colo.) and the publicly available lukF-PV and lukS-PV gene sequences from S. aureus (GenBank accession no. AB006796, X72700, AB009866, and AB045978) as described by Ryan and McDonald., J Clin Microbiol, 2005; 43:6147-6149. Any of the PVL probes or primers described by Ryan and McDonald may be used in the present invention.

With the present invention, genomic DNA for PCR analysis may be prepared by any of a variety of methods. For example, extraction by a standard procedure such as that described in Ausubel, F. M., R. Brent, R. E. Kingston, B. D. Moore, J. G. Seidman, J. A. Smith, and K. Struhl. 1987. Current protocols in molecular biology. Greene Publishing Associates and Wiley Interscience, New York, N.Y. may be used.

The methods of the present invention include analyzing a methicillin-resistant S. aureus bacterium for the presence or absence of an AT repeat region in the conserved hypothetical gene SACOL0058. An AT repeat region having at least 6 (also referred to herein as “greater than or equal to 6,” “6 or more,” and “≧6”) AT repeats in the conserved hypothetical gene SACOL0058 is found in the USA300 strain of methicillin-resistant S. aureus.

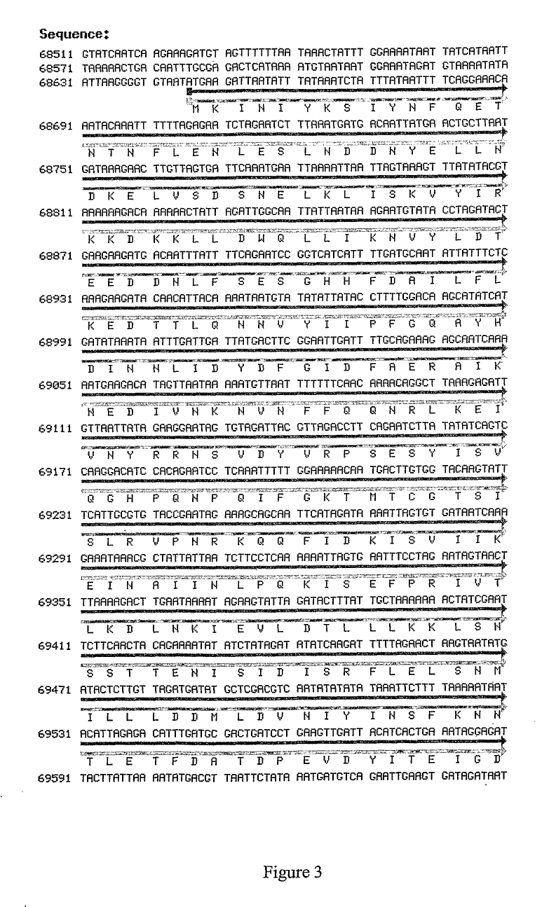

The complete genome sequence of S. aureus strain COL is available (Genebank Accession Number CP000046). Within this genome sequence, located about 1.4 kb beyond the J1 SCCmec-chromosomal junction, is the conserved hypothetical gene SACOL0058. The conserved hypothetical gene SACOL0058 is in a chromosomal region flanking the SCCmec element. The complete nucleotide sequence of the conserved hypothetical gene SACOL0058 and its hypothetical amino acids sequence are shown in FIG. 3 and can be found at Genebank Accession No. NC_{—}002951 and on the worldwide web at ncbi.nlm.nih.gov/entrez/viewer.fcgi?db=nucleotide&dopt=graph&_from=61796&_to=77239&val=NC_{—}002951. The conserved hypothetical gene SACOL0058 in S. aureus strain COL includes within it nucleotides 69954 to 70855 of Genebank Accession Number CP000046. The present invention demonstrates that only strain USA300 isolates contain a sequence of ≧6 AT repeats in combination with the presence of the PVL toxin gene.

Any of a variety of techniques may be used to analysis a methicillin-resistant S. aureus bacterium for the presence or absence of an AT repeat region in the conserved hypothetical gene SACOL0058 and a determination of the number of AT repeats present. For example, nucleic acid based hybridization assays, PCR-based assays, or sequencing based assays may be used.

In a preferred embodiment, analyzing a methicillin-resistant S. aureus bacterium for the presence or absence of a an AT repeat region in the conserved hypothetical gene SACOL0058 and a determination of the number of AT repeats present is undertaken by performing PCR with oligonucleotide primers capable of amplifying the AT repeat region and analyzing for the presence or absence of amplified DNA fragments of the AT repeat region. In some embodiments, the resultant amplified DNA product(s) may be sequenced.

A variety of primers may be selected that flank the AT repeat region in the conserved hypothetical gene SACOL0058, based on the genomic sequence of this region. In a preferred embodiment, oligonucleotide primers may be chosen that will only amplify the AT repeat region when 6 or more AT repeats are present. Such primers may have one or more locked nucleic acid (LNA) oligonucleotides incorporated therein. One or more LNA bases may incorporated into the forward primer. The forward oligonucleotide primer may include the sequence TGCTCGACGTCAATATATATATAT (SEQ ID NO:7) or variations thereof. The LNA bases may be placed at any one or more of the nucleotides of this sequence. For example, in one embodiment, the forward oligonucleotide primer may have the sequence TG^{L}CT^{L}CGA^{L}CGTCAA^{L}TA^{L}T ATATATAT (SEQ ID NO:5), wherein N^{L} represents a locked nucleic acid base. The reverse primer may include the sequence 5′-ACGATGATATTCC CGATAG-3′ (SEQ ID NO:8) or 5′-CAATTAACGATGATATTCCCGATAG-3′ (SEQ ID NO:4).

Locked nucleic acid (LNA) is a novel type of nucleic acid analog that contains a 2′-O, 4′-C methylene bridge. This bridge restricts the flexibility of the ribofuranose ring and locks the structure into a rigid bicyclic formation, conferring enhanced hybridization performance and exceptional biostability. Duplexes including LNA oligonucleotides are considerably more thermally stable that similar duplexes constituted from DNA or RNA oligonucleotides. LNA oligonucleotides form a thermodynamically stable primer with improved target specificity under stringent annealing conditions (see, for example, Vester and Wengel, Biochemistry 2004; 43:13233-13241; McTigue et al., Biochemistry, 2004; 43:5388-5404; Jensen et al., J. Chem. Soc., Perkin Trans, 2001; 2:1224-1232; Christensen et al., Biochem. J., 2001; 354:481-484 (2001); and on the worldwide web at proligo.com). LNAs for use in the synthesis of oligonucleotides are commercially available, for example, from Proligo LLC (Boulder, Colo.). Standard DNA synthesizer platforms can be used for the synthesis of oligonucleotides including LNAs and no change is required in the reagents commonly used for DNA synthesis and LNAs can be applied to most platforms that employ synthetic oligonucleotides.

In some embodiments, the present invention further includes analyzing methicillin-resistant S. aureus DNA for the presence or absence of at least a portion of the conserved hypothetical gene SACOL0058, the sequence of which is shown in FIG. 3. Any of a variety of techniques may be used to analysis a methicillin-resistant S. aureus bacterium for the presence or absence of the conserved hypothetical gene SACOL0058. For example, nucleic acid based hybridization assays, PCR-based assays, restriction mapping, or sequencing based assays may be used. In a preferred embodiment, analyzing a methicillin-resistant S. aureus bacterium for the presence or absence of at least a portion of the conserved hypothetical gene SACOL0058 is undertaken by performing PCR with oligonucleotide primers capable of amplifying at least a portion of the conserved hypothetical gene SACOL0058. In some embodiments, the resultant amplified DNA product(s) may be sequenced. A variety of primers may be selected, including, but no limited to, ATreg-1, 5′-GAAAATGGAATAGAG TTGGCAGAC-3′ (SEQ ID NO:3) and ATreg-2, 5′-CAATTAACGATGATA TTCCCGATAG-3′ (SEQ ID NO:4).

The present invention includes isolated oligonucleotide primers for use in the methods of the present invention. For, example, the present invention includes, but is not limited to, any of the oligonucleotide primers described herein, including, oligonucleotide primers having the sequence 5′-ATCATTAGGTAAAATGTCTGGACATGATCCA-3′ (SEQ ID NO:1); 5′-GCATCAACTGTATTGGATAGCAAAAGC-3′ (SEQ ID NO:2); 5′-ACGATGATATTCCCGATAG-3′ (SEQ ID NO:3); 5′-CAATTAACGATGATATTCCCGATAG-3′ (SEQ ID NO:4); 5′-TGCTCGACGTCAATATATATATAT (SEQ ID NO:7) and 5′-ACGATGATATTCCCGATAG-3′ (SEQ ID NO:8).

The present invention includes an oligonucleotide primer with the sequence 5′-TGCTCGACGTCAATATATATATAT (SEQ ID NO:7), wherein one or more of the bases are a locked nucleic acid base. In a preferred embodiment, the oligonucleotide primer is TG^{L}CT^{L}CGA^{L}CGTCAA^{L}TA^{L}T ATATATAT (SEQ ID NO:5) wherein N^{L} represents a locked nucleic acid base.

The present invention also includes kits including an oligonucleotide primer pair capable of amplifying an AT repeat region of in the conserved hypothetical gene SACOL0058 of S. aureus DNA and an oligonucleotide primer capable of amplifying a gene for the PVL toxin. In some embodiments, the kit may also include a primer pair capable of amplifying at least a portion of the conserved hypothetical gene SACOL0058.

With the present invention, the analysis of a methicillin-resistant S. aureus bacterium for the presence or absence of a gene for the Panton-Valentine Leukocidin (PVL) toxin, the presence or absence of an AT repeat region in the conserved hypothetical gene SACOL0058; and the presence or absence the conserved hypothetical gene SACOL0058 may be performed in any of a variety of formats. For example, one or more of these analyses may be performed separately, may be performed as a multiplex reaction, in a single reaction vessel, or may be performed as a microarray. In some embodiments, a methicillin-resistant S. aureus bacterium may be analyzed for the presence or absence of additional markers.

Samples that can be used in the methods of the present invention can be obtained from any source including, but not limited to, blood, blood products, tissue, ascites, culture media, body fluids, skin, pus, urogenital specimens, feces, foodstuffs, beverages, cosmetic products, pharmaceutical products, healthcare products, surfaces such as floors and tables, and airborne particles such as pollen and dust. A sample may be obtained from a clinical isolates, for example, and isolate obtained from skin or soft tissue infections. A sample may be obtained from a swab of a body site, for example, from the nose, including, but not limited to, the anterior nares, the throat, the perineum, the axilla, or the skin. A sample may be one that is suspected of having microorganisms, in particular, S. aureus. The sample may already have been tested for the presence of microorganisms and have tested positive for microorganisms.

The present invention includes an isolated DNA fragment of a methicillin-resistant USA300 S. aureus bacterium gene, wherein the fragment includes an AT repeat region of the conserved hypothetical gene SACOL0058 having 6 or more AT repeats. Such an isolated DNA fragment may have 6, 7, 8, 9, 10, or more AT repeats. Such an isolated DNA fragment may have 6 to 8 AT repeats. As used herein, the term “isolated” means that a polynucleotide is either removed from its natural environment or synthetically derived, for instance by recombinant techniques, or chemically or enzymatically synthesized. An isolated polynucleotide denotes a polynucleotide that has been removed from its natural genetic milieu and is thus free of other extraneous or unwanted coding sequences, and is in a form suitable for use within genetically engineered protein production systems. Isolated polynucleotides of the present invention are free of other coding sequences with which they are ordinarily associated, but may include naturally occurring 5′ and 3′ untranslated regions such as promoters and terminators. Preferably, the polynucleotide is purified, i.e., essentially free from any other polynucleotides or polypeptides and associated cellular products or other impurities.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Rapid Multiplex PCR Assay for Identification of USA300 Community-Acquired Methicillin-Resistant Staphylococcus aureus (CA-MRSA) Isolates

Recent reports have noted a discernible increase in the number of community-acquired methicillin-resistant Staphylococcus aureus (CA-MRSA) infections in patients without traditional risk factors. In the United States, the most prominent CA-MRSA strain encodes Panton-Valentine leukocidin (PVL) cytotoxin genes, belongs to pulsed field gel electrophoresis (PFGE) type USA300, multi-locus sequence type 8 (ST8), and carries staphylococcal cassette chromosome (SCC) mec type IV. At present, molecular characterization of MRSA such as USA300 can be time consuming and is often beyond the technical capability of many clinical laboratories, making routine identification difficult. With the present example, the chromosomal regions flanking the SCCmec element in 44 USA300 MRSA isolates were analyzed. A signature ‘AT repeat’ sequence was identified within the conserved hypothetical gene SACOL0058 located 1.4 kilobases (kb) downstream of the 3′ end of the J1 SCCmec-chromosomal junction. Only USA300 isolates contained a sequence of ≧6 AT repeats in combination with PVL. Related USA500 or Iberian strains had ≧6 AT repeats but were PVL negative. Using a locked nucleic acid (LNA) primer specific for ≧6 AT repeats in combination with primers to detect PVL, a multiplex PCR assay specific for the identification of USA300 strains was developed. Multiplex results were 100% concordant with DNA sequencing, indicating the usefulness of the method has promise as a means of rapidly identifying these problem isolates.

### Materials and Methods

Bacterial strains. A total of 106 S. aureus strains (105 MRSA, 1 MSSA) were examined in this study (Table 1). Strains were chosen according to MLST and PFGE type, with 44 belonging to the USA300. Of these, 30 isolates belonged to USA300-0114 clonal complex 8 (CC8:ST8) (Enright et al., Proc. Natl. Acad. Sci. U.S.A 2002; 99:7687-7692); all where independent isolates known to be epidemiologically unrelated. Other isolates were included on the basis of their genetic relatedness to USA300 strains as determined by MLST BURST analysis (available on the worldwide web at //mlst.net).

Pulsed field gel electrophoresis. All strains were analyzed by PFGE. Bacterial DNA was prepared according to the rapid protocol of Goering (Goering, 1993. Pulsed Field Gel Electrophoresis, p. 185-196. In D. H. Persing, T. F. Smith, F. C. Tenover, and T. J. White (eds.), Molecular Microbiology: Diagnostic Principles and Practice. ASM Press, Washington, D.C.). Pulsed field patterns were analyzed using BioNumerics software (v. 4.6, Applied Maths, Kortrijk, Belgium) according to published criteria (McDougal et al., “Pulsed-field gel electrophoresis typing of oxacillin-resistant Staphylococcus aureus isolates from the United States: establishing a national database,” J. Clin Microbiol. 2003; 41:5113-5120).

PCR. Chromosomal DNA was isolated for PCR using the method described by Enright et al. on the MLST website (available on the worldwide web at //saureus.mlst.net/misc/info.asp; see also, Enright et al., J Clin Microbiol, 2003; 38:1008-1015).

Detection of PVL genes was performed using primers luk-PV-1, 5′-ATCATTAGGTAAAATGTCTGGACATGATCCA-3′ (SEQ ID NO:1) and luk-PV-2, 5′-GCATCAACTGTATTGGATAGCAAAAGC-3′ (SEQ ID NO:2), generating a 433 basepair (bp) product as described by Lina et al. (Lina et al., Clin Infect Dis, 19199; 29:1128-1132).

Primer sequences used to detect the conserved hypothetical gene SACOL0058 (S. aureus strain COL, Genebank accession number CP000046) were ATreg-1, 5′-GAAAATGGAATAGAGTTGGCAGAC-3′ (SEQ ID NO:3) and ATreg-2, 5′-CAATTAACGATGATATTCCCGATAG-3′ (SEQ ID NO:4), resulting in an amplification product of 902 bp. Reaction mixtures (100 μl total volume) contained 1.5 mM MgCl₂, 200 μM dNTP mix, primers at a final concentration of 0.5 mM, 2.5 U Taq DNA polymerase (Roche Diagnostics, Mannheim, Germany), and 1 μl (ca. 1 μg) of template DNA. Amplification was carried out for 34 cycles with denaturation at 94° C. for 30 seconds, annealing at 66° C. for 30 seconds, extension at 72° C. for 1 minute 30 seconds, and final extension at 72° C. for five minutes.

The primer designed to discriminate the number of AT repeats present within SACOL0058 was lnaAT-5′-TG^{L}CT^{L}GCA^{L}CGTCAA^{L}TA^{L}TAT ATATAT-3′ (SEQ ID NO:5) (nucleotides 69490-69511, designed with an additional AT at the 3′ end of the primer). Locked nucleic acid oligonucleotides (LNA; Sigma-Proligo, Boulder, Colo.) within the primer are indicated by (^{L}) (Vester and Wengel, Biochemistry 2004; 43:13233-13241). This primer was coupled with the ATreg-2 primer with PCR conditions as described above for detection of SACOL0058, but with an annealing temperature of 67° C. for 30 seconds and 5 U of Taq polymerase yielding a product of 1,366 bp in size.

Multiplex PCR, to simultaneously detect PVL, SACOL0058, and the number of AT repeats, was performed as for SACOL0058, but with primers at the following concentrations: luk-PV-1, luk-PV-2, and ATreg-1 at 0.05 μM, ATreg-2 at 0.75 μM, lnaAT at 0.5 μM, and 5 U of Taq DNA polymerase per reaction.

Amplification reactions were visualized by agarose gel electrophoresis (1.5% SeaKem LE [FMC BioProducts, Rockland, Me.]) in 1× Tris-borate-EDTA (TBE) buffer.

PCTG products were sequenced at the Creighton University Molecular Biology Core Facility using an ABI Prism® 3100 Avant Genetic Analyzer (Applied Biosystems, Foster City, Calif.).

### Results

Identification of Signature USA300 AT-repeat sequence. The SCCmec chromosomal region in MRSA isolates is known to be recombinogenic, resulting in a variety of SCCmec types (Hanssen and Ericson Sollid, FEMS Immunol Med Microbiol, 2006; 46:8-20). This example focused on USA300-specific sequences in genomic regions directly flanking SCCmec, reasoning that these areas might also be subject to higher rates of recombination.

Using a variety of primers, analysis of ca. 3 kb of genomic sequence upstream of the orfX side of SCCmec revealed 100% homology across all 30 USA300-0114 (CC8:ST8) strains studied. In addition, the sequence was similar to seven non-USA300 Staphylococcus aureus genomes (MW2, COL, Mu50, N315, NCTC8325, MRSA252, and MSSA476) (see the worldwide web at ncbi.nlm.nih.gov). However, analysis of 3.3 Kb of genomic sequence extending beyond the J1-SCCmec-chromosomal junction (Ito et al., Drug Resist. Updat, 2003; 6:41-52) revealed a region containing either 6 or 8 repetitions of an adenine-thymine base pair in all USA300 isolates examined. Sequence comparison with S. aureus strain COL (CC8:ST250) located the AT-repeat region approximately 1.4 kb downstream from the J1-SCCmec-chromosomal junction, within the conserved hypothetical gene SACOL0058. However, SACOL0058 contained only 5 AT repeats in COL. In addition, PCR analysis reveled the presence of SACOL0058 in the PVL-negative USA100, USA500, and USA800 isolates. SACOL0058 was absent from the chromosome of the prototypical community-associated USA400 MRSA strain MW2 (CC1:ST1)

Locked nucleic acid PCR to detect the presence and extent of the AT-repeat sequence. Traditional oligonucleotide primers were not suitable for AT-repeat detection due to the potential of the multiple 3′ repeats to facilitate in hairpin formation, primer dimers, etc. Therefore, LNA oligonucleotides were used to ensure correct hybridization and discrimination between 5 and ≧6 AT repeats. This specificity results from the fact that LNA oligonucleotides are modified with a 2′-O, 4′-C methylene bridge forming a thermodynamically stable primer with improved target specificity under stringent annealing conditions (Vester and Wengel. 2004. Biochemistry 43:13233-13241).

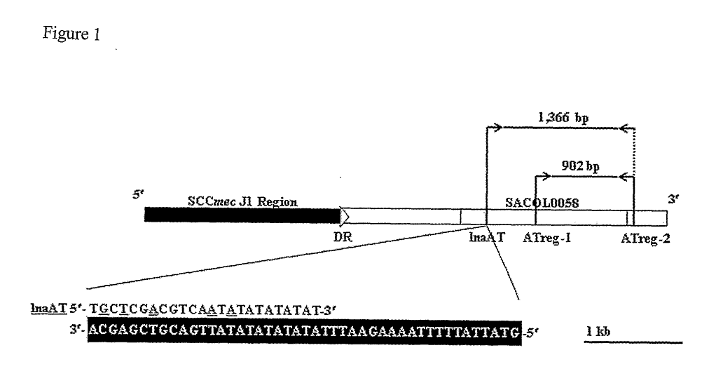

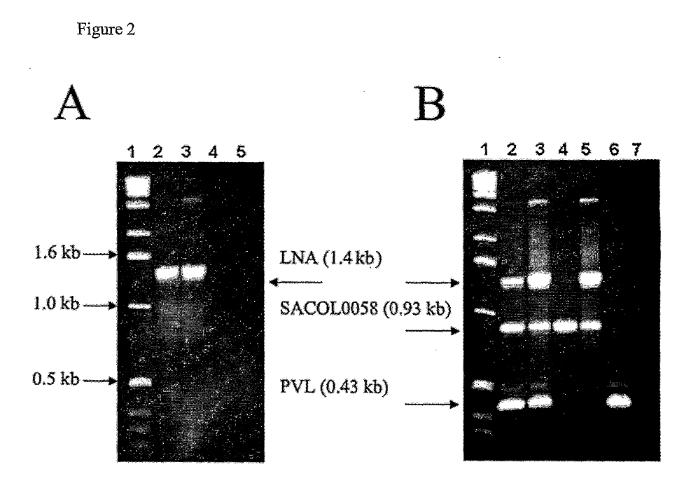

A LNA-PCR primer was designed with 6 AT repeats at the 3′ end and LNA-modified bases near the 5′ end to strongly drive correct hybridization and PCR amplification when used with an appropriate reverse primer in isolates with ≧6 AT repeats (FIG. 1). As shown in FIG. 2a, amplification was observed only in strains containing ≧6 (i.e., 6 or 8) AT repeats. These results were 100% concordant with DNA sequence analysis. LNA-PCR was used to examine a variety of strains belonging to MLST clonal complexes CC5 and CC8, including single locus variants of both groups, and double or triple locus variants of CC8. In addition to USA300:ST8 isolates, SACOL0058 was found in CC8:ST247 (Iberian clone), CC8:ST250 (USA500), CC5:ST5 (USA100 and USA800) and CC5:ST228 isolates, but was absent from CC8:ST239 and CC8:ST240 isolates of the Brazilian clone. However, as noted previously, only USA300 isolates, which carry PVL genes, possessed either 6 or 8 AT repeats. Results for all isolates examined are shown on Table 1.

Multiplex PCR for the rapid identification of USA300 PFT isolates. Using minor modification of the PCR reaction, primers were combined to create a multiplex PCR assay with the potential to differentiate USA300 isolates from other MRSA strains. As shown in FIG. 2b, lanes 2 and 3, USA300 strains were identified by three PCR products: (i) SACOL0058, (ii) LNA based amplification of ≧6 AT repeats, and (iii) PVL genes. A USA500:ST8 isolate, containing 6 AT repeats, was distinguished from the USA300-0114 isolates by the absence of the PVL band (FIG. 2, lane 4). The mecA-negative isolate, CRG-1112 (NCTC8325), was a positive control for SACOL0058 (FIG. 2b, lane 4) while strain CRG-956 (USA400, MW2) served as a positive control for PVL (FIG. 2b, lane 6). Discrepant amplification products, when observed, were not problematic due to their minor intensity and size variation.

TABLE 1
S. aureus isolate characteristics

| Isolate | MLST^{a} | PFT^{b}/Strain Designation | PVL | AT Repeat^{c} | Source |
|---|---|---|---|---|---|
| 956 | CC1:ST1 | USA400 (MW2) | + | ND | CDC^{d} |
| CH17 | CC1:ST1^{e} | USA400 | + | ND | This study |
| CH18 | CC1:ST1^{e} | USA400 | + | ND | This study |
| CH33 | CC1:ST1^{e} | USA400 | − | ND | This study |
| CH62 | CC1:ST1^{e} | USA400 | + | ND | This study |
| CH48 | CC1:ST1^{e} | USA400 | + | ND | This study |
| 947 | CC5:ST5 | USA100 (N315) | − | 5 | CDC |
| 948 | CC5:ST5 | USA100 (Mu50) | − | 5 | CDC |
| 1244 | CC5:ST5 | USA800 | − | ≦5 | CDC |
| 1245 | CC5:ST5 | USA800 | − | ≦5 | CDC |
| 1226 | CC5:ST228 | Denmark 3727-03 | − | ≦5 | SSI^{f} |
| 1112 | CC8:ST8 | NCTC8325^{g} | − | 5 | CDC |
| 1116 | CC8:ST8 | USA300-0114^{h} | + | 6 | CDC |
| 1117 | CC8:ST8 | USA300-0114 | + | 6 | CDC |
| 1126 | CC8:ST8 | USA300-0114 | + | 6 | CDC |
| 1127 | CC8:ST8 | USA300-0114 | + | 6 | CDC |
| 1128 | CC8:ST8 | USA300-0114 | + | 6 | CDC |
| 1129 | CC8:ST8 | USA300-0114 | + | 8 | CDC |
| 1130 | CC8:ST8 | USA300-0114 | + | 8 | CDC |
| 1131 | CC8:ST8 | USA300-0114 | + | 6 | CDC |
| 1132 | CC8:ST8 | USA300-0114 | + | 8 | CDC |
| 1133 | CC8:ST8 | USA300-0114 | + | 6 | CDC |
| 1134 | CC8:ST8 | USA300-0114 | + | 6 | CDC |
| 1135 | CC8:ST8 | USA300-0114 | + | 6 | CDC |
| 1136 | CC8:ST8 | USA300-0114 | + | 6 | CDC |
| 1137 | CC8:ST8 | USA300-0114 | + | 6 | CDC |
| 1138 | CC8:ST8 | USA300-0114 | + | 6 | CDC |
| 1139 | CC8:ST8 | USA300-0114 | + | 6 | CDC |
| 1140 | CC8:ST8 | USA300-0114 | + | 8 | CDC |
| 1141 | CC8:ST8 | USA300-0114 | + | 6 | CDC |
| 1142 | CC8:ST8 | USA300-0114 | + | 6 | CDC |
| 1143 | CC8:ST8 | USA300-0114 | + | 6 | CDC |
| 1144 | CC8:ST8 | USA300-0114 | + | 6 | CDC |
| 1145 | CC8:ST8 | USA300-0114 | + | 8 | CDC |
| 1146 | CC8:ST8 | USA300-0114 | + | 6 | CDC |
| 1147 | CC8:ST8 | USA300-0114 | + | 6 | CDC |
| 1148 | CC8:ST8 | USA300-0114 | + | 6 | CDC |
| 1149 | CC8:ST8 | USA300-0114 | + | 6 | CDC |
| 1150 | CC8:ST8 | USA300-0114 | + | 6 | CDC |
| 1151 | CC8:ST8 | USA300-0114 | + | 6 | CDC |
| 1152 | CC8:ST8 | USA300-0114 | + | 6 | CDC |
| 1233 | CC8:ST8 | USA300-0114 | + | ≧6 | CDC |
| 1235 | CC8:ST8 | USA300-0045ⁱ | + | ≧6 | CDC |
| 1335 | CC8:ST8^{e} | USA300-0045ⁱ | + | + | CDC |
| 1330 | CC8:ST8^{e} | USA300-0068^{j} | + | + | CDC |
| 1118 | CC8:ST8 | USA300-0120^{k} | + | 6 | CDC |
| 1119 | CC8:ST8 | USA300-0120 | + | 6 | CDC |
| 1234 | CC8:ST8 | USA300-0120 | + | ≧6 | CDC |
| 1236 | CC8:ST8 | USA300-0120 | + | 6 | CDC |
| 1336 | CC8:ST8^{e} | USA300-0120 | + | + | CDC |
| 1333 | CC8:ST8^{e} | USA300-0120 | + | + | CDC |
| 1334 | CC8:ST8^{e} | USA300-0120 | + | + | CDC |
| 1337 | CC8:ST8^{e} | USA300-0120ⁱ | + | + | CDC |
| 1237 | CC8:ST8 | USA300-0247^{h} | + | 6 | CDC |
| 1238 | CC8:ST8 | USA300-0251^{h} | + | ≧6 | CDC |
| 1331 | CC8:ST8^{e} | USA300-0272^{l} | + | + | CDC |
| 1228 | CC8:ST8 | USA500 | − | ≧6 | CDC |
| 1230 | CC8:ST8 | USA500 | − | 6 | CDC |
| 1231 | CC8:ST8 | USA500 | − | 6 | CDC |
| 511 | CC8:ST239 | Brazilian | − | ND | CDC |
| 513 | CC8:ST239 | Brazilian | − | ND | CDC |
| 515 | CC8:ST239 | Brazilian | − | ND | CDC |
| 517 | CC8:ST239 | Brazilian | − | ND | CDC |
| 519 | CC8:ST239 | Brazilian | − | ND | CDC |
| 521 | CC8:ST239 | Brazilian | − | ND | CDC |
| 523 | CC8:ST239 | Brazilian | − | ND | CDC |
| 525 | CC8:ST239 | Brazilian | − | ND | CDC |
| 1186 | CC8:ST239 | Brazilian | − | ND | CDC |
| 1187 | CC8:ST239 | Brazilian | − | ND | CDC |
| 1188 | CC8:ST239 | Brazilian | − | ND | CDC |
| 1189 | CC8:ST239 | Brazilian | − | ND | CDC |
| 1191 | CC8:ST239 | Brazilian | − | ND | CDC |
| 1192 | CC8:ST239 | Brazilian | − | ND | CDC |
| 1184 | CC8:ST239 | Brazilian | − | ND | CDC |
| 1190 | CC8:ST239 | Brazilian | − | ND | CDC |
| 1225 | CC8:ST239 | Denmark 2731-03 | − | ND | SSI |
| 1185 | CC8:ST240 | Brazilian | − | ND | CDC |
| 510 | CC8:ST247 | Iberian | − | ≦5 | CDC |
| 512 | CC8:ST247 | Iberian | − | ≦5 | CDC |
| 514 | CC8:ST247 | Iberian | − | ≦5 | CDC |
| 516 | CC8:ST247 | Iberian | − | ≦5 | CDC |
| 518 | CC8:ST247 | Iberian | − | ≦5 | CDC |
| 520 | CC8:ST247 | Iberian | − | ≦5 | CDC |
| 522 | CC8:ST247 | Iberian | − | ≦5 | CDC |
| 524 | CC8:ST247 | Iberian | − | ≦5 | CDC |
| 1227 | CC8:ST247 | Iberian | − | 5 | CDC |
| 1229 | CC8:ST247 | Iberian | − | 5 | CDC |
| 1241 | CC8:ST247 | Iberian | − | ≦5 | CDC |
| 930 | CC8:ST250 | USA500 (COL) | − | 5 | CDC |
| 1246 | CC15:ST15 | USA900 | − | ND | CDC |
| 1220 | CC22:ST22 | Denmark 40135 | − | ND | SSI |
| 1221 | CC22:ST22 | Denmark 22744-99 | − | ND | SSI |
| 1224 | CC22:ST22 | Denmark 848-03 | − | ND | SSI |
| 1232 | CC22:ST22 | EMRSA15 | − | ND | CDC |
| 1172 | CC30:ST30 | USA200/EMRSA16 | − | ND | SMRSAL^{m} |
| 1248 | CC30:ST30 | USA1100 | + | ND | CDC |
| 958 | CC30:ST30 | USA200 | − | ND | SMRSAL |
| 1223 | CC30:ST36 | Denmark 2551-03 | − | ND | SSI |
| 1222 | CC30:ST579 | Denmark 1391-03 | − | ND | SSI |
| 1242 | CC45:ST45 | USA600 | − | ND | CDC |
| 1247 | CC59:ST59 | USA1000 | + | ND | CDC |
| 1243 | CC72:ST72 | USA700 | − | ND | CDC |
| 1197 | CC80:ST80 | Denmark 188851-95 | + | ND | SSI |
| 1198 | CC80:ST80 | Denmark 11819-97 | + | ND | SSI |
| 1212 | CC80:ST80 | France HT0401 | + | ND | SSI |
| 1214 | CC80:ST80 | Greece 14 | + | ND | SSI |

^{a}Clonal complex:Sequence type.
^{b}Pulsed field type as described by McDougal et al. (23).
^{c}ND = Not Detected; 5, 6, or 8 = number of AT repeats confirmed with sequencing; ≦5 or ≧6 = number of AT repeats detected via PCR assay.
^{d}Centers for Disease Control and Prevention, Atlanta, Georgia, USA.
^{e}MLST type according to PFGE pattern.
^{f}Robert Skov - Statens Serum Institute, Copenhagen, Denmark.
^{g}Methicillin-susceptible Staphylococcus aureus.
^{h}SCCmec type IVa. USA300-0114 were independent isolates from known geographic locations known to be epidemiologically unrelated.
ⁱSCCmec type IVb,
^{j}SCCmec type non-typable, not IVa, b or c.
^{k}USA300-0120 isolates (except 1337) were SCCmec type IVb.
^{l}SCCmec type IVc.
^{m}Donald Morrison - Scottish MRSA Reference Laboratory, Glasgow, UK.

### Discussion

USA300 CA-MRSA are a clear and emerging clinical concern. However, the definitive identification of these strains has traditionally involved a combination of tests and protocols (i.e., PFGE, MLST, SCCmec, PVL) which require specialized expertise and several days to complete. In addition, the newly described arginine catabolic mobile element (ACME) recently described by Diep et al. (Diep et al., Lancet 2006; 367:731-739) appears to only be present in USA300 strains carrying SCCmec type IVa (McDougal et al., Abstr. 46th Intersci. Conf. Antimicrob. Agents Chemother., abstr. C2-603, 2006). The multiplex assay described here differentiates USA300 CA-MRSA strains with a variety of SCCmec IV subtypes (see Table 1) from other MRSA. In this example, only USA300 isolates contained either 6 or 8 AT repeats as well as PVL genes. In some instances, isolates with related sequence types such as USA500 (ST8) exhibited ≧6 AT repeats, but never in combination with PVL. Other isolates (e.g., ST80) encoded PVL but always contained <6 AT repeats. Thus, the combined detection of these elements via multiplex PCR allowed USA300 isolates to be quickly and specifically identified without sequencing. As with any assay, variant strains may exist that could be difficult to detect with this method. Nevertheless, the results of this example demonstrate the potential of the LNA assay as a rapid, cost-effective approach for identifying USA300 CA-MRSA, a significant pathogen with increasing prevalence in many hospital and community settings.

In the CC8 isolates examined, SACOL0058 was present in ST8, ST247, 1 247, and ST250 but not in ST239 and ST240, consistent with MLST analysis as discussed by Enright et al. (Enright et al., Proc. Natl. Acad. Sci. U.S.A 2002; 99:7687-7692). Interestingly, SACOL0058 was also found in CC5.

Protein sequence analysis of conserved hypothetical gene SACOL0058 via the Accelrys GCG® Translation program (San Diego, Calif.) showed MRSA strains containing 5 AT repeats may possess a fully functional protein. However, an additional AT repeat (i.e., 6 AT repeats) resulted in a reading frame shift producing a stop codon at amino acid 286. With 8 AT repeats, the first 285 amino acids of the protein remain homologous to the original with divergence thereafter. While the data presented in this example do not address questions regarding the functional role for SACOL0058 in staphylococcal isolates, the region appears to remain conserved among strains especially including the USA300 genotype.

AT-repeat PCR, in combination with PCR for the presence of PVL genes and the SACOL0058, has the potential to identify USA300 CA-MRSA strains in a rapid, cost efficient manner. Accurate results can be obtained by carefully following optimized PCR conditions, allowing valuable diagnostic and surveillance data to be collected quickly without the need for sequencing.

The words “preferred” and “preferably” refer to embodiments of the invention that may afford certain benefits, under circumstances. However, other embodiments may also be preferred, under the same or other circumstances. Furthermore, the recitation of one or more preferred embodiments does not imply that other embodiments are not useful, and is not intended to exclude other embodiments from the scope of the invention.

Also herein, the recitation of numerical ranges by endpoint includes all numbers subsumes within that range (e.g. 1 to 5 includes 1, 1.5, 2, 2.75, 3, 3.8, 4, 5, etc.).

The above description of the invention is not intended to describe each disclosed embodiments of every implementation of the present invention; rather, only illustrative embodiments are described.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

For any method disclosed herein that includes discrete steps, the steps may be conducted in any feasible order. And, as appropriate, any combination of two or more steps may be conducted simultaneously.

SEQUENCE FREE LISTING

| | |
|---|---|
| SEQ ID NO: 1-4, | Synthetic oligonucleotide primers |
| 7, 8 | |
| SEQ ID NO: 5 | sequence of synthetic oligonucleotide 1naAT primer |
| SEQ ID NO: 6 | genomic sequence of AT repeat region in a USA300 |
| | MRSA strain |
| SEQ ID NO: 9 | nucleotides sequence of SACOL0058 |
| SEQ ID NO: 10 | hypothetical protein sequence of SACOL0058 |

1. A method for identifying the USA300 strain of methicillin-resistant S. aureus, the method comprising:
analyzing a methicillin-resistant S. aureus bacterium for the presence or absence of a gene for the Panton-Valentine Leukocidin (PVL) toxin; and
analyzing methicillin-resistant S. aureus DNA for the presence or absence of an AT repeat region in the conserved hypothetical gene SACOL0058;
wherein the presence of a gene for the PVL toxin and the presence of an AT repeat region comprising at least 6 AT repeats in the conserved hypothetical gene SACOL0058 indicates that the methicillin-resistant S. aureus bacterium is the USA300 strain of methicillin-resistant S. aureus.

2. The method of claim 1 wherein analyzing methicillin-resistant S. aureus DNA for the presence or absence of an AT repeat region in the conserved hypothetical gene SACOL0058 comprises:
performing a polymerase chain reaction with oligonucleotide primers capable of amplifying an AT repeat region in the conserved hypothetical gene SACOL0058 if present; and
analyzing for the presence or absence of amplified DNA fragments containing an AT repeat region.

3. The method of claim 2 wherein the oligonucleotide primers will only amplify the AT repeat region when 6 or more AT repeats are present.

4. The method of claim 3 wherein the oligonucleotide primers comprise a forward primer having one or more locked nucleic acid bases incorporated therein.

5. The method of claim 4 wherein the forward oligonucleotide primer has the sequence TGCTCGACGTCAATATATATATAT (SEQ ID NO:7) wherein one or more of the nucleic acids is a locked nucleic acid base.

6. The method of claim 5 wherein the forward oligonucleotide primer has the sequence TG^{L}CT^{L}CGA^{L}CGTCAA^{L}TA^{L}TATATATAT (SEQ ID NO:5) wherein N^{L} represents a locked nucleic acid base.

7. The method of claim 3 wherein the oligonucleotide primers comprise a reverse primer having the sequence 5′-ACGATGATATTCCCGATAG-3′ (SEQ ID NO:8) or 5′-CAATTAACGATGATATTCCCGATAG-3′ (SEQ ID NO:4).

8. The method of claim 2 further comprising sequencing the amplified DNA fragments.

9. The method of claim 1 wherein analyzing a methicillin-resistant S. aureus bacterium for the presence or absence of a gene for the PVL toxin comprises:
performing a polymerase chain reaction with oligonucleotide primers capable of amplifying a gene for the PVL toxin; and
analyzing for the presence or absence of amplified DNA fragments of a gene for the PVL toxin.

10. The method of claim 9 wherein the oligonucleotide primers capable of amplifying a gene for the PVL toxin comprise 5′-ATCATTAGGTAAAATGTCTGGACATGATCCA-3′ (SEQ ID NO:1) and 5′-GCATCAACTGTATTGGATAGCAAAAGC-3′ (SEQ ID NO:2).

11. The method of claim 9 further comprising sequencing the amplified DNA fragments.

12. The method of claim 1 wherein a methicillin-resistant S. aureus bacterium other than the USA300 strain has less than 6 AT repeats and/or no gene for the PVL toxin.

13. The method of claim 1 wherein analyzing a methicillin-resistant S. aureus bacterium for the presence or absence of a gene for the Panton-Valentine Leukocidin (PVL) toxin and analyzing methicillin-resistant S. aureus DNA for the presence or absence of an AT repeat region in the conserved hypothetical gene SACOL0058 occurs in a single-vessel experiment.

14. The method of claim 1 wherein analyzing methicillin-resistant S. aureus DNA for the presence or absence of an AT repeat region in the conserved hypothetical gene SACOL0058 comprises:
performing a polymerase chain reaction with oligonucleotide primers capable of amplifying an AT repeat region in the conserved hypothetical gene SACOL0058 if present; and
analyzing for the presence or absence of amplified DNA fragments containing an AT repeat region; and
wherein analyzing a methicillin-resistant S. aureus bacterium for the presence or absence of a gene for the PVL toxin comprises:
performing a polymerase chain reaction with oligonucleotide primers capable of amplifying a gene for the PVL toxin; and
analyzing for the presence or absence of amplified DNA fragments of a gene for the PVL toxin.

15. The method of claim 14 further comprising analyzing methicillin-resistant S. aureus DNA for the presence or absence of the conserved hypothetical gene SACOL0058, wherein analyzing methicillin-resistant S. aureus DNA for the presence or absence of the conserved hypothetical gene SACOL0058 comprises:
performing a polymerase chain reaction with oligonucleotide primers capable of amplifying an at least a portion of the conserved hypothetical gene SACOL0058; and
analyzing for the presence or absence of amplified DNA fragments containing at least a portion of the conserved hypothetical gene SACOL0058.

16. The method of claim 14 wherein performing the polymerase chain reactions occurs in a single-vessel experiment.

17. An isolated DNA fragment of a methicillin-resistant USA300 S. aureus bacterium gene, wherein the fragment includes an AT repeat region of the conserved hypothetical gene SACOL0058 comprising 6 or more AT repeats.

18. The isolated DNA fragment of claim 17 wherein there are 6-8 AT repeats in the AT repeat region.

19. An isolated oligonucleotide primer having the sequence TG^{L}CT^{L}CGA^{L}CGTCAA^{L}TA^{L}TATATATAT (SEQ ID NO:5) wherein N^{L} represent a locked nucleic acid base.

20. An isolated oligonucleotide primers selected from the group consisting of 5′-ACGATGATATTCCCGATAG-3′ (SEQ ID NO:3) and 5′-CAATTAACGATGATATTCCCGATAG-3′ (SEQ ID NO:4).

21. A kit comprising an oligonucleotide primer pair capable of amplifying an AT repeat region of in the conserved hypothetical gene SACOL0058 of S. aureus DNA and an oligonucleotide primer capable of amplifying a gene for the PVL toxin.

22. The kit of claim 21 further comprising a primer pair capable of amplifying at least a portion of the conserved hypothetical gene SACOL0058.


SEQUENCE LISTING (12 sequences)

SEQ ID NO: 1 - DNA, 31 bp, ARTIFICIAL SEQUENCE
  oligonucleotide primer
atcattaggtaaaatgtctggacatgatcca

SEQ ID NO: 2 - DNA, 27 bp, ARTIFICIAL SEQUENCE
  Oligonucleotide Primer
gcatcaactgtattggatagcaaaagc

SEQ ID NO: 3 - DNA, 24 bp, ARTIFICIAL SEQUENCE
  Oligonucleotide Primer
gaaaatggaatagagttggcagac

SEQ ID NO: 4 - DNA, 25 bp, ARTIFICIAL SEQUENCE
  Oligonucleotide Primer
caattaacgatgatattcccgatag

SEQ ID NO: 5 - DNA, 24 bp, ARTIFICIAL SEQUENCE
  Oligonucleotide Primers with modified bases 1, 3, 6, 12, and 14 “locked nucleic acid”
tgctcgacgtcaatatatatatat

SEQ ID NO: 6 - DNA, 23 bp, ARTIFICIAL SEQUENCE
  Oligonucleotide Primer with modified bases 2, 4, 7, 13, and 15 “locked nucleic acid”
gctcgacgtcaatatatatatat

SEQ ID NO: 7 - DNA, 24 bp, ARTIFICIAL SEQUENCE
  Oligonucleotide Primer
tgctcgacgtcaatatatatatat

SEQ ID NO: 8 - DNA, 19 bp, ARTIFICIAL SEQUENCE
  Oligonucleotide Sequence
acgatgatattcccgatag

SEQ ID NO: 9 - DNA, 1140 bp, STAPHYLOCOCCUS AUREUS
gtatcaatcaagaaagatgtagttttttaataaactatttggaaaataattatcataatttaaaaactgacaatttgcgagactcataaaatgtaataatggaaatagatgtaaaatataattaaggggtgtaatatgaagattaatatttataaatctatttataattttcaggaaacaaatacaaattttttagagaatctagaatctttaaatgatgacaattatgaactgcttaatgataaagaacttgttagtgattcaaatgaattaaaattaattagtaaagtttatatacgtaaaaaagacaaaaaactattagattggcaattattaataaagaatgtatacctagatactgaagaagatgacaatttattttcagaatccggtcatcattttgatgcaatattatttctcaaagaagatacaacattacaaaataatgtatatattataccttttggacaagcatatcatgatataaataatttgattgattatgacttcggaattgattttgcagaaagagcaatcaaaaatgaagacatagttaataaaaatgttaatttttttcaacaaaacaggcttaaagagattgttaattatagaaggaatagtgtagattacgttagaccttcagaatcttatatatcagtccaaggacatccacagaatcctcaaatttttggaaaaacaatgacttgtggtacaagtatttcattgcgtgtaccgaatagaaagcagcaattcatagataaaattagtgtgataatcaaagaaataaacgctattattaatcttcctcaaaaaattagtgaatttcctagaatagtaactttaaaagacttgaataaaatagaagtattagatactttattgctaaaaaaactatcgaattcttcaactacagaaaatatatctatagatatatcaagatttttagaactaagtaatatgatactcttgttagatgatatgctcgacgtcaatatatatataaattcttttaaaaataatacattagagacatttgatgcgactgatcctgaagttgattacatcactgaaataggagattacttattaaaatatgacgttaattctataaatgatgtcagaattgaagtgatagataat

SEQ ID NO: 10 - protein, 315 aa, STAPYLOCOCCUS AUREUS
MKINIYKSIYNFQETNTNFLENLESLNDDNYELLNDKELVSDSNELKLISKVYIRKKDKKLLDWQLLIKNVYLDTEEDDNLFSESGHHFDAILFLKEDTTLQNNVYIIPFGQAYHDINNLIDYDFGIDFAERAIKNEDIVNKNVNFFQQNRLKEIVNYRRNSVDYVRPSESYISVQGHPQNPQIFGKTMTCGTSISLRVPNRKQQFIDKISVIIKEINAIINLPQKISEFPRIVTLKDLNKIEVLDTLLLKKLSNSSTTENISIDISRFLELSNMILLLDDMLDVNIYINSFKNNTLETFDATDPEVDYITEIGD

SEQ ID NO: 11 - DNA, 24 bp, ARTIFICIAL SEQUENCE
  Oligonucleotide Primer
tgctcgacgtcaatatatatatat

SEQ ID NO: 12 - DNA, 45 bp, STAPHYLOCOCCUS AUREUS
acgagctgcagttatatatatatatttaagaaaatttttattatg